(19) United States
(12) Patent Application Publication
(10) Pub. No.: US 2025/0189501 A1
(43) Pub. Date: Jun. 12, 2025

(54) HYDROGEN SENSOR AND HYDROGEN DETECTION METHOD

(71) Applicant: TDK CORPORATION, Tokyo (JP)

(72) Inventor: Tomoyuki SASAKI, Tokyo (JP)

(73) Assignee: TDK CORPORATION, Tokyo (JP)

(21) Appl. No.: 18/975,134

(22) Filed: Dec. 10, 2024

Related U.S. Application Data
(60) Provisional application No. 63/608,944, filed on Dec. 12, 2023.

(51) Int. Cl. G01N 33/00; G01N 27/72

(52) U.S. Cl. CPC G01N 33/0062; G01N 27/72; G01N 33/005

(57) ABSTRACT

A hydrogen sensor includes: a substrate; an underlying layer with conductivity; and a hydrogen adsorption layer with a resistance changing in response to adsorption of hydrogen. The underlying layer is located between the substrate and the hydrogen adsorption layer. A surface roughness of the hydrogen adsorption layer is smaller than a film thickness of the hydrogen adsorption layer.

### BACKGROUND

### Field

The present disclosure relates to a hydrogen sensor and a hydrogen detection method.

### Description of Related Art

Hydrogen has attracted attention as one energy source. On the other hand, hydrogen is a combustible gas that causes an explosion even at a low concentration by bonding to oxygen. It is said that the lower explosion limit concentration of hydrogen is about 4%. In order to manage a hydrogen concentration in a space, a hydrogen sensor capable of detecting a hydrogen concentration has been required.

As sensors adapted to detect hydrogen, a semiconductor-type sensor, a contact combustion-type sensor, an electrochemical sensor, a thermal conduction-type sensor, and the like are known. Each of the sensors has pros and cons. For example, Patent Document 1 discloses that it is possible to implement a hydrogen sensor with a wide measurement range by using a plurality of types of sensors of different detection schemes.

### PATENT DOCUMENTS

[Patent Document 1] Japanese Unexamined Patent Application, First Publication No. 2020-30137

### SUMMARY OF THE INVENTION

Operation currents of the semiconductor-type sensor, the contact combustion-type sensor, the electrochemical sensor, the thermal conduction-type sensor, and the like are large, and a large amount of power is thus consumed. Also, operation temperatures of these sensors are as high as several hundreds of degrees or more, and it is difficult to stably perform measurement in an environment at room temperature.

The present disclosure was made in view of the aforementioned circumstances, and an object thereof is to provide a hydrogen sensor with a small operation current that can be used even at room temperature, and a hydrogen detection method using the hydrogen sensor.

The present disclosure provides the following mechanisms in order to solve the aforementioned problem.

A hydrogen sensor according to a first aspect includes: a substrate; an underlying layer with conductivity; and a hydrogen adsorption layer with a resistance changing in response to adsorption of hydrogen. The underlying layer is located between the substrate and the hydrogen adsorption layer. A surface roughness of the hydrogen adsorption layer is smaller than a film thickness of the hydrogen adsorption layer.

### BRIEF DESCRIPTION OF THE DRAWINGS

FIG. 1 is a plan view of a hydrogen sensor according to a first embodiment.

FIG. 2 is a sectional view of the hydrogen sensor according to the first embodiment.

FIG. 3 is a sectional view of a hydrogen sensor according to a second embodiment.

FIG. 4 is a sectional view of a hydrogen sensor according to a third embodiment.

FIG. 5 is a plan view of a hydrogen sensor according to a fourth embodiment.

FIG. 6 is a plan view of a hydrogen sensor according to a fifth embodiment.

FIG. 7 is a plan view of a hydrogen sensor according to a sixth embodiment.

FIG. 8 is a sectional view of a sensor element in the hydrogen sensor according to the sixth embodiment.

FIG. 9 is a sectional view of a magnetic resistance effect element in the hydrogen sensor according to the sixth embodiment.

FIG. 10 is a diagram for explaining a function of the hydrogen sensor according to the sixth embodiment.

### DETAILED DESCRIPTION OF THE INVENTION

Hereinafter, embodiments will be described in detail with reference to the drawings as needed. The drawings used in the following description may illustrate characteristic parts in an enlarged manner for convenience in order to promote understanding of features, and dimensional ratios of components may differ from those in practice. Materials, dimensions, and the like described as examples in the following description are only examples, and the present disclosure is not limited thereto and can be appropriately modified and implemented within a scope in which effects of the present disclosure are achieved.

First, directions will be defined. One direction on a plane in which each layer spreads is defined as an X direction, and a direction perpendicularly intersecting the X direction is defined as a Y direction. A direction perpendicularly intersecting the X direction and the Y direction is defined as a Z direction. The Z direction is a stacking direction of each layer. Although a +Z direction from a substrate toward a hydrogen adsorption layer may be represented as “up” and a −Z direction which is opposite to the +Z direction may be represented as “down” in the specification, these expressions are adopted for convenience and are not intended to define the direction of gravity.

Also, “extending in the X direction” means that the dimension in the X direction is larger than the minimum dimension out of the dimensions in the X direction, the Y direction, and the Z direction, for example, in the specification. The same also applies to cases where components extend in other directions.

### First Embodiment

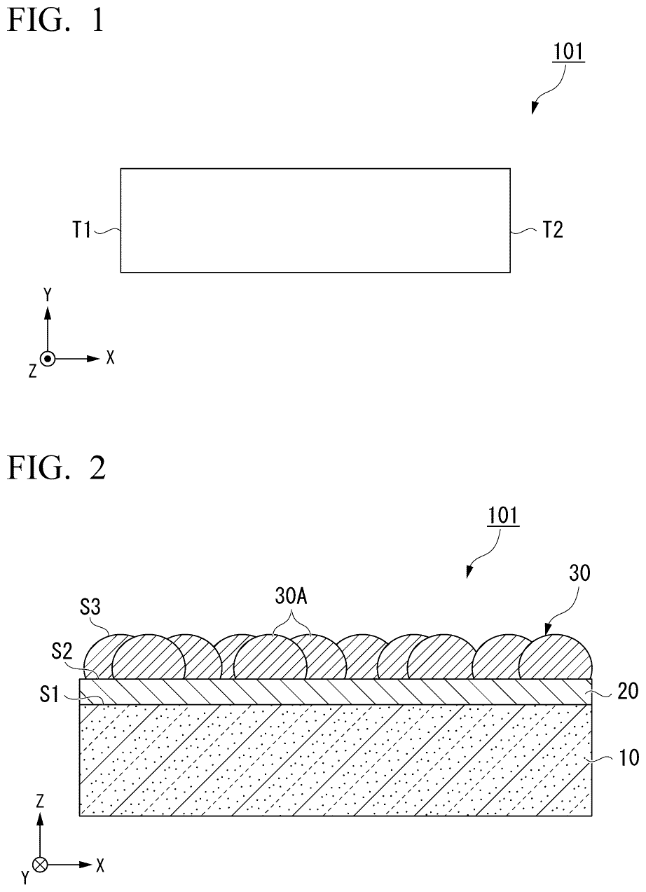

FIG. 1 is a plan view of a hydrogen sensor 101 according to a first embodiment. FIG. 2 is a sectional view of the hydrogen sensor 101 according to the first embodiment.

The hydrogen sensor 101 detects the amount of hydrogen in a measurement environment by measuring a change in resistance between a first terminal T1 and a second terminal T2. The resistance between the first terminal T1 and the second terminal T2 can be obtained by Ohm's law by applying a current between the first terminal T1 and the second terminal T2. The hydrogen sensor 101 can detect hydrogen within a range of equal to or greater than 10 ppm and equal to or less than 10 vol %, for example.

The current to be applied when the resistance is measured may be a direct current or an alternating current. When an alternating current is used to measure the resistance, it is possible to curb migration of elements inside a hydrogen adsorption layer 30 and to curb breakage of the hydrogen sensor 101. The amount of current to be applied when the resistance is measured is, for example, equal to or less than 10 mA.

The hydrogen sensor 101 may have any shape in a plan view. As illustrated in FIG. 1, for example, the length of the hydrogen sensor 101 in a first direction (the X direction in FIG. 1) connecting the first terminal T1 and the second terminal T2 is preferably longer than the length thereof in a second direction (the Y direction in FIG. 1) perpendicularly intersecting the first direction. When the length of the hydrogen sensor 101 in a current flowing direction is long, the amount of hydrogen to be adsorbed increases, and the amount of change in resistance of the hydrogen sensor 101 increases.

The shape of the hydrogen sensor 101 in a plan view is not limited to a rectangular shape illustrated in FIG. 1 and may be a meander wiring structure or a zigzag wiring structure. The meander wiring structure is a structure in which wirings meander.

The hydrogen sensor 101 includes a substrate 10, an underlying layer 20, and a hydrogen adsorption layer 30.

The substrate 10 is a support body of the underlying layer 20 and the hydrogen adsorption layer 30. The substrate 10 is a semiconductor or an insulator.

The underlying layer 20 is located between the substrate 10 and the hydrogen adsorption layer 30. The underlying layer 20 has conductivity. The underlying layer 20 is a conductive film spreading in a first surface S1 of the substrate 10. The first surface S1 is a surface of the substrate 10 on the side of the underlying layer 20.

The resistivity of the underlying layer 20 is higher than the resistivity of the hydrogen adsorption layer 30, for example. A change in resistance in the hydrogen sensor 101 occurs in the hydrogen adsorption layer 30. When the resistivity of the underlying layer 20 is high, a largest part of the current for observing a change in resistance flows into the hydrogen adsorption layer 30, and sensitivity of the hydrogen sensor 101 is enhanced.

The underlying layer 20 contains any one selected from a group consisting of NiCr, β-W, Zr, Cr, Ta, alumel, chromel, Ti, NbN, TaN, TiN, VN, and ZrN, for example.

The film thickness of the underlying layer 20 is, for example, equal to or less than 3 nm. The film thickness of the underlying layer 20 is preferably equal to or greater than 0.4 nm, for example. The film thickness of the underlying layer 20 is an average value of film thicknesses at ten different measurement points in an XY plane. The film thickness of the underlying layer 20 can be obtained from a section image of a scanning electron microscope (SEM), for example. As the film thickness of the underlying layer 20 decreases, a larger part of current for observing a change in resistance flows into the hydrogen adsorption layer 30, and the sensitivity of the hydrogen sensor 101 is further enhanced.

The hydrogen adsorption layer 30 is stacked on a first surface S2 of the underlying layer 20. The first surface S2 is a surface of the underlying layer 20 on a side far from the substrate 10. The hydrogen adsorption layer 30 is in contact with the underlying layer 20, for example. A surface S3 of the hydrogen adsorption layer 30 is exposed to outside air. The surface S3 of the hydrogen adsorption layer 30 is a surface of the hydrogen adsorption layer 30 on the side opposite to a surface on which the hydrogen adsorption layer 30 is in contact with the underlying layer 20.

A surface roughness of the surface S3 of the hydrogen adsorption layer 30 is smaller than the film thickness of the hydrogen adsorption layer 30. The surface roughness of the surface S3 of the hydrogen adsorption layer 30 is larger than a surface roughness of the first surface S1 of the substrate 10, for example. The surface roughness is an arithmetic mean roughness Ra. Since the surface roughness of the hydrogen adsorption layer 30 is smaller than the film thickness, the hydrogen adsorption layer 30 is a continuous film spreading in the first surface S2 of the underlying layer 20. If the surface S3 of the hydrogen adsorption layer 30 includes unevenness, the surface area of the hydrogen adsorption layer 30 increases, and sensitivity of the hydrogen sensor 101 is enhanced.

The film thickness of the hydrogen adsorption layer 30 is, for example, less than 2 nm. The film thickness of the hydrogen adsorption layer 30 is preferably equal to or greater than 0.4 nm, for example. The film thickness of the hydrogen adsorption layer 30 is an average value of film thicknesses at ten different measurement points in the XY plane. If the film thickness of the hydrogen adsorption layer 30 is sufficiently thin, the surface S3 of the hydrogen adsorption layer 30 is not sufficiently flattened by a deposited material, and the surface S3 becomes uneven.

The hydrogen adsorption layer 30 may include a plurality of particles 30A. The plurality of particles 30A are in close contact with each other. The plurality of particles 30A are dispersed in the XY plane when seen in the Z direction. If the hydrogen adsorption layer 30 includes the plurality of particles 30A, unevenness is formed on the surface S3 of the hydrogen adsorption layer 30, and sensitivity of the hydrogen sensor 101 is enhanced.

The hydrogen adsorption layer 30 is a layer with a resistance changing in response to adsorption of hydrogen. The hydrogen adsorption layer 30 has conductivity.

A hydrogen adsorption material constituting the hydrogen adsorption layer 30 is metal, an alloy, an oxide, a nitride, or an oxynitride containing any one selected from a group consisting of Mg, Al, Ti, Fe, Zr, Pd, Pt, Hf, and Ta. If hydrogen is adsorbed by such a hydrogen adsorption material, an electron state or a crystal structure of the hydrogen adsorption layer 30 changes, and the resistance changes. The hydrogen adsorption material constituting the hydrogen adsorption layer 30 preferably contains Pt.

In a case where the hydrogen adsorption layer 30 contains Pd, for example, hydrogen adsorbed by the hydrogen adsorption layer 30 is inserted between Pd atoms and changes the electron state and the crystal structure of the hydrogen adsorption layer 30. In the case where the hydrogen adsorption layer 30 contains Pd, a change in resistance in the hydrogen sensor 101 when hydrogen is adsorbed is large.

In a case where the hydrogen adsorption layer 30 contains Pt, for example, hydrogen adsorbed by the hydrogen adsorption layer 30 is not inserted between Pt atoms and changes an electron state of the hydrogen adsorption layer 30. In the case where the hydrogen adsorption layer 30 contains Pt, the crystal structure of the hydrogen adsorption layer 30 does not change, and the hydrogen sensor 101 is unlikely to be degraded. Also, in the case where the hydrogen adsorption layer 30 contains Pt, hydrogen adsorbed by the hydrogen adsorption layer 30 is not inserted between the Pt atoms, and it is thus possible to detect adsorption of hydrogen even when a hydrogen concentration is high.

Examples of a preferable combination between the underlying layer 20 and the hydrogen adsorption layer 30 include a combination of β-W of 2 nm as the underlying layer 20 and Pt of 2 nm as the hydrogen adsorption layer.

The hydrogen sensor 101 can be produced by forming films of the layers and working the stacked films into a predetermined shape. For the film formation for the layers, it is possible to use, for example, a sputtering method, a chemical vapor deposition method (CVD method), an electron beam evaporation method (EB evaporation method), and an atomic laser deposition method. Each layer can be worked using photolithography, for example.

Then, a hydrogen concentration detection method of the hydrogen sensor 101 will be described. The hydrogen adsorption layer 30 of the hydrogen sensor 101 is exposed to a measurement environment. Hydrogen contained in the measurement environment is adsorbed by the hydrogen adsorption layer 30.

If hydrogen is adsorbed by the hydrogen adsorption layer 30, the electron state or the crystal structure of the hydrogen adsorption layer 30 changes. A change in state of the hydrogen adsorption layer 30 leads to a change in resistance of the hydrogen adsorption layer 30.

If a current is caused to flow between the first terminal T1 and the second terminal T2 of the hydrogen sensor 101, a resistance of the hydrogen sensor 101 is obtained by Ohm's law. A relationship between the resistance of the hydrogen sensor 101 and the hydrogen concentration in the measurement environment is measured in advance. It is possible to obtain the hydrogen concentration in the measurement environment from the resistance of the hydrogen sensor 101 on the basis of the result.

Also, in a case where the hydrogen concentration in the measurement environment decreases, hydrogen is released from the hydrogen adsorption layer 30 to the measurement environment. Released hydrogen reacts with oxygen in the surroundings and becomes water. Therefore, an influence of released hydrogen on the hydrogen concentration in the measurement environment is small.

If water adheres to the hydrogen adsorption layer 30, hydrogen is unlikely to be adsorbed by the hydrogen adsorption layer 30, and the sensitivity of the hydrogen sensor 101 decreases. It is possible to evaporate water adhering to the hydrogen adsorption layer 30 by causing a pulse current to flow through the hydrogen sensor 101 and causing the hydrogen sensor 101 to generate heat, for example. In addition to this, a heater or the like that causes water adhering to the hydrogen adsorption layer 30 to evaporate may be separately provided. In order to prevent oxygen and hydrogen from bonding to each other, a gas barrier film that does not allow oxygen to penetrate therethrough may be installed in the surroundings of the hydrogen sensor 101.

The hydrogen sensor 101 according to the present embodiment measures the amount of hydrogen in the measurement environment on the basis of a change in resistance due to adsorption of hydrogen to the hydrogen adsorption layer 30. Since the change in resistance can be measured with a small amount of current, the hydrogen sensor 101 according to the present embodiment consumes a small amount of power. Also, it is possible to further reduce the amount of current necessary to detect the change in resistance by reducing the thickness of each layer, and to further reduce power consumed by the hydrogen sensor 101.

The hydrogen sensor 101 according to the present embodiment also operates at room temperature. The hydrogen sensor 101 according to the present embodiment does not require heating or the like of the element at the time of measurement, and a measurement speed thereof is also high.

Moreover, the resistance of the hydrogen sensor 101 also changes in response to adsorption of methane or ethane. Performance of detecting methane or ethane is about 1/100 the performance of detecting hydrogen. Therefore, an influence of methane or ethane is sufficiently ignorable even in a case where hydrogen and methane or ethane are present at the same time in the measurement environment and in the environment at room temperature.

On the other hand, the hydrogen sensor 101 can also function as a sensor adapted to detect methane or ethane in a case where methane or ethane is present in the measurement environment and in an environment at a high temperature.

Also, the substrate 10 is a support body and does not significantly affect the function of the sensor, the substrate 10 may be removed from the hydrogen sensor 101.

### Second Embodiment

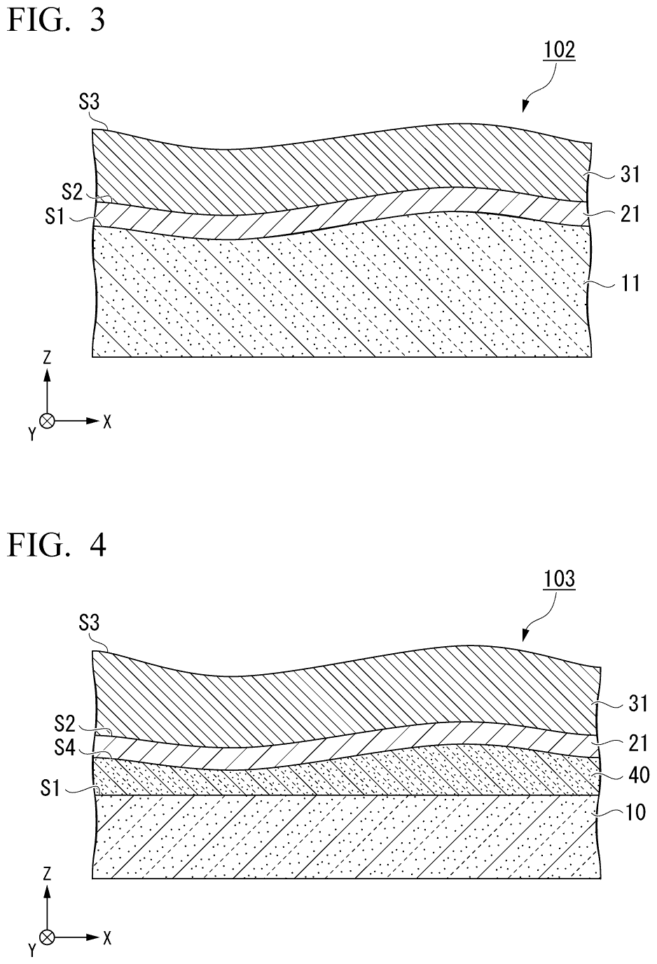

FIG. 3 is a sectional view of a hydrogen sensor 102 according to a second embodiment. The hydrogen sensor 102 includes a substrate 11, an underlying layer 21, and a hydrogen adsorption layer 31. The shape of the hydrogen sensor 102 in a plan view is similar to that of the hydrogen sensor 101. An operation principle of the hydrogen sensor 102 is also similar to that of the hydrogen sensor 101.

The substrate 11 is different from the substrate 10 in that a first surface S1 of the substrate 11 includes unevenness. The other points of the substrate 11 are the same as those of the substrate 10. The unevenness is formed by the first surface S1 projecting or recessed in the Z direction with respect to a plane that is parallel to the XY plane.

The underlying layer 21 is different from the underlying layer 20 in that a first surface S2 of the underlying layer 21 includes unevenness. The other points of the underlying layer 21 are the same as those of the underlying layer 20. The first surface S2 of the underlying layer 21 is curved along the first surface S1 of the substrate 11.

The hydrogen adsorption layer 31 is different from the hydrogen adsorption layer 30 in that the hydrogen adsorption layer 31 is a continuous film which does not include a plurality of particles. The other points of the hydrogen adsorption layer 31 are similar to those of the hydrogen adsorption layer 30. A surface roughness of the hydrogen adsorption layer 31 is smaller than the film thickness of the hydrogen adsorption layer 31. A surface S3 of the hydrogen adsorption layer 31 is curved along the first surface S1 of the substrate 11 and the first surface S2 of the underlying layer 21. The surface area of the hydrogen adsorption layer 30 increase and sensitivity of the hydrogen sensor 102 is enhanced by the surface S3 being curved.

The hydrogen sensor 102 according to the second embodiment have effects similar to those of the hydrogen sensor 101 according to the first embodiment. The hydrogen adsorption layer 31 may be a single continuous film, and the unevenness of the surface S3 may be originated from the first surface S1 of the substrate 11.

### Third Embodiment

FIG. 4 is a sectional view of a hydrogen sensor 103 according to a third embodiment. The hydrogen sensor 103 includes a substrate 10, an intermediate layer 40, an underlying layer 21, and a hydrogen adsorption layer 31. The shape of the hydrogen sensor 103 in a plan view is similar to that of the hydrogen sensor 101. An operation principle of the hydrogen sensor 103 is also similar to that of the hydrogen sensor 101. Configurations in the hydrogen sensor 103 similar to those in the hydrogen sensor 101 and the hydrogen sensor 102 will be denoted by similar reference signs, and description thereof will be omitted.

The intermediate layer 40 is located between the substrate 10 and the underlying layer 21. A material constituting the intermediate layer 40 is similar to the material constituting the substrate 10, for example. A first surface S4 of the intermediate layer 40 includes unevenness. The unevenness is formed by the first surface S4 projecting or recessed in the Z direction with respect to a plane that is parallel to the XY plane. A first surface S2 of the underlying layer 21 and a surface S3 of the hydrogen adsorption layer 31 are curved along the first surface S4 of the intermediate layer 40.

The hydrogen sensor 103 according to the third embodiment have effects similar to those of the hydrogen sensor 101 according to the first embodiment. The curve of the surface S3 of the hydrogen adsorption layer 31 may be formed by the first surface S4 of the intermediate layer 40.

### Fourth Embodiment

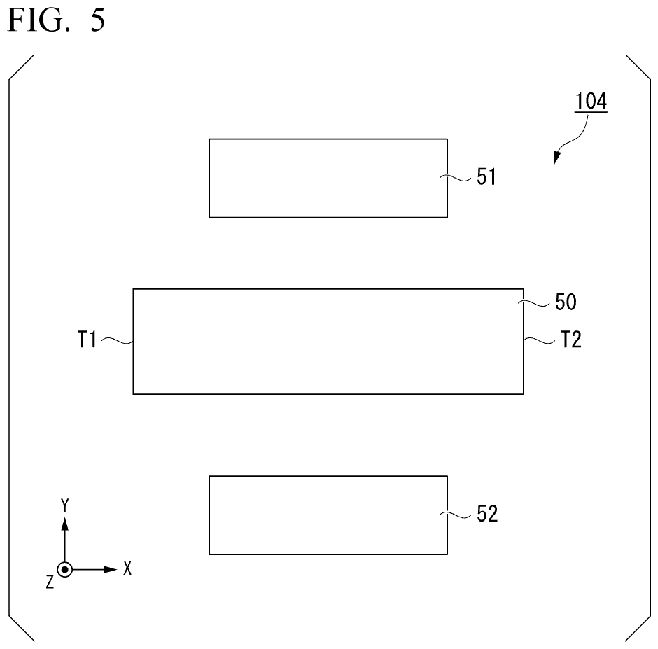

FIG. 5 is a plan view of a hydrogen sensor 104 according to a fourth embodiment. The hydrogen sensor 104 includes a sensor element 50, a heat absorber 51, and a heat generator 52.

The sensor element 50 includes a substrate, an underlying layer, and a hydrogen adsorption layer. The sensor element 50 may be similar to the hydrogen sensor 101 according to the first embodiment, may be similar to the hydrogen sensor 102 according to the second embodiment, or may be similar to the hydrogen sensor 103 according to the third embodiment.

The heat absorber 51 is located inside the same measurement environment as that of the sensor element 50 and at a position where the heat absorber 51 is not in a direct electrical contact with the sensor element 50. The heat absorber 51 absorbs heat from the measurement environment. It is only necessary for the heat absorber 51 to absorb heat, and the heat absorber 51 may be a metal plate as a single member or may be a Peltier element, for example. Once the heat absorber 51 absorbs heat, heat convection occurs in the measurement environment, and air in the measurement environment is circulated. The sensor element 50 can accurately detect the amount of hydrogen in the measurement environment by the air in the measurement environment being circulated.

The heat generator 52 is located in the same measurement environment as that of the sensor element 50 and at a position where the heat generator 52 is not in a direct electrical contact with the sensor element 50. The heat generator 52 provides heat to the measurement environment. It is only necessary for the heat generator 52 to cause heat, and the heat generator 52 is, for example, a heater. Once the heat generator 52 generates heat, heat convection occurs in the measurement environment, and air in the measurement environment is circulated. The sensor element 50 can accurately detect the amount of hydrogen in the measurement environment by the air in the measurement environment being circulated.

The hydrogen sensor 104 according to the fourth embodiment has effects similar to those of the hydrogen sensor 101 according to the first embodiment. Also, the hydrogen sensor 104 can cause the air inside the measurement environment to be circulated by causing heat convection in the measurement environment, and can accurately detect the amount of hydrogen in the measurement environment. Although the example in which the heat absorber 51 and the heat generator 52 are used at the same time in the hydrogen sensor 104 has been described, only any one of them may be used.

### Fifth Embodiment

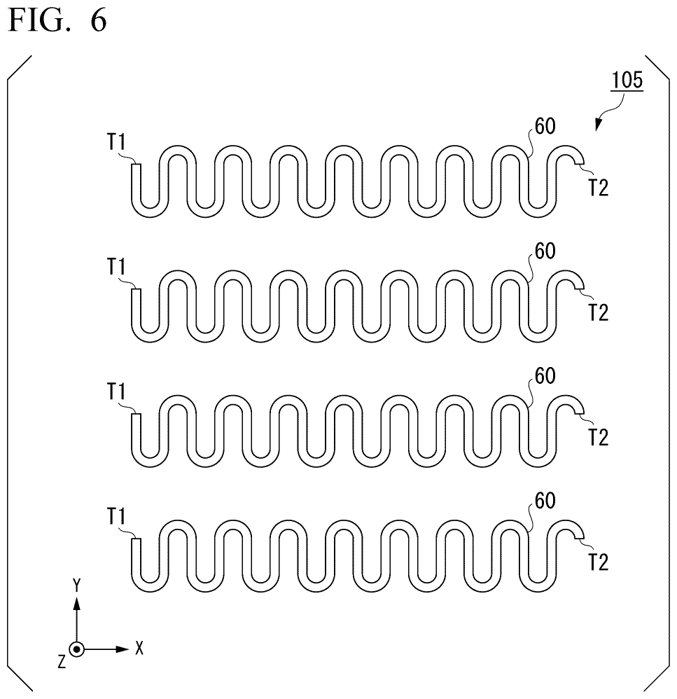

FIG. 6 is a plan view of a hydrogen sensor 105 according to a fifth embodiment. The hydrogen sensor 105 according to the fifth embodiment includes a plurality of sensor elements 60.

Each of the sensor elements 60 includes a substrate, an underlying layer, and a hydrogen adsorption layer. The layer structure of the sensor element 60 may be similar to that in the hydrogen sensor 101 according to the first embodiment, may be similar to that in the hydrogen sensor 102 according to the second embodiment, or may be similar to that in the hydrogen sensor 103 according to the third embodiment.

The sensor elements 60 are located at different positions in the same measurement environment. The shape of each sensor element 60 in a plan view is a meander wiring structure. The area in which hydrogen can be adsorbed increases, and the amount of change in resistance of the sensor element 60 can be increased, by causing the sensor element 60 to meander.

The hydrogen sensor 105 according to the fifth embodiment has effects similar to those of the hydrogen sensor 101 according to the first embodiment. The hydrogen sensor 105 according to the fifth embodiment can measure a distribution of hydrogen concentrations in the measurement environment by including the plurality of sensor elements 60.

The hydrogen sensor 105 according to the fifth embodiment may also be provided with the heat absorber 51 and the heat generator 52 according to the fourth embodiment. Also, the shape of each sensor element 60 in a plan view is not limited to the meander wiring structure and may be any shape.

### Sixth Embodiment

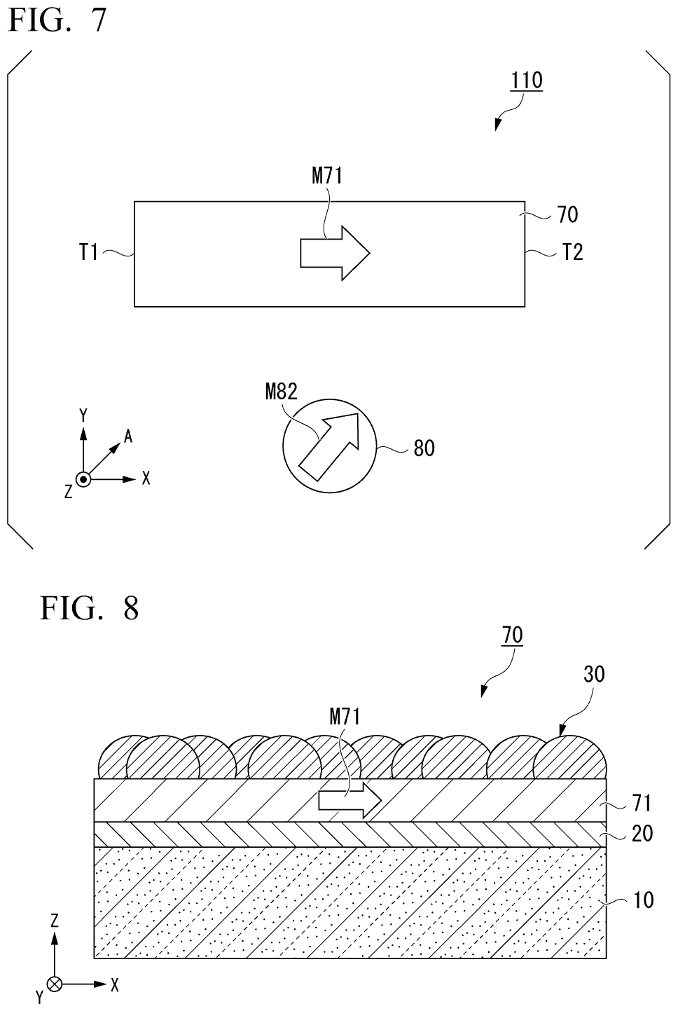

FIG. 7 is a plan view of a hydrogen sensor 110 according to a sixth embodiment. The hydrogen sensor 110 according to the sixth embodiment includes a sensor element 70 and a magnetic resistance changing element 80.

The sensor element 70 may have any shape in a plan view. As illustrated in FIG. 7, for example, the length of the sensor element 70 in a first direction (the X direction in FIG. 7) connecting a first terminal T1 and a second terminal T2 is preferably longer than the length thereof in a second direction (the Y direction in FIG. 7) perpendicularly intersecting the first direction. The shape of the sensor element 70 in a plan view is not limited to that in the example in FIG. 7 and may be a meander wiring structure or a zigzag wiring structure.

FIG. 8 is a sectional view of the sensor element 70 in the hydrogen sensor 110 according to the sixth embodiment. The sensor element 70 includes a substrate 10, an underlying layer 20, a magnetic layer 71, and a hydrogen adsorption layer 30. The sensor element 70 is different from the hydrogen sensor 101 according to the first embodiment in that the sensor element 70 includes the magnetic layer 71.

The substrate 10, the underlying layer 20, and the hydrogen adsorption layer 30 are similar to those in the hydrogen sensor 101 according to the first embodiment. The substrate 10, the underlying layer 20, and the hydrogen adsorption layer 30 in the sensor element 70 may be replaced with the hydrogen sensor 102 according to the second embodiment or the hydrogen sensor 103 according to the third embodiment.

The magnetic layer 71 is a ferromagnetic body. The ferromagnetic body is, for example, metal selected from a group consisting of Cr, Mn, Co, Fe, and Ni, an alloy containing one or more kinds of such metal, an alloy containing such metal and at least one or more kinds of elements from B, C, and N, or the like. The ferromagnetic body is, for example, Co—Fe, Co—Fe—B, Ni—Fe, a Co—Ho alloy, an Sm—Fe alloy, an Fe—Pt alloy, a Co—Pt alloy, or a CoCrPt alloy.

The magnetic layer 71 includes a magnetization M71. In FIGS. 7 and 8, the orientation direction of the magnetization M71 in a state where an external magnetic field is not applied is indicated by the arrows. Although FIGS. 7 and 8 illustrate an example in which the magnetization M71 is orientated in the X direction, the orientation direction of the magnetization M71 is not limited to the example. For example, the magnetization M71 may be orientated in any direction within the XY plane or may be orientated in the Z direction.

The magnetic resistance changing element 80 is located in the same measurement environment as that of the sensor element 70 and at a position where the magnetic resistance changing element 80 is not in a direct electrical contact with the sensor element 70. The magnetic resistance changing element 80 is any one selected from a group consisting of an anisotropic magneto resistance (AMR) element, a giant magneto resistance (GMR) element, a tunnel magneto resistance (TMR) element, and an anomalous Hall effect (AHE) element.

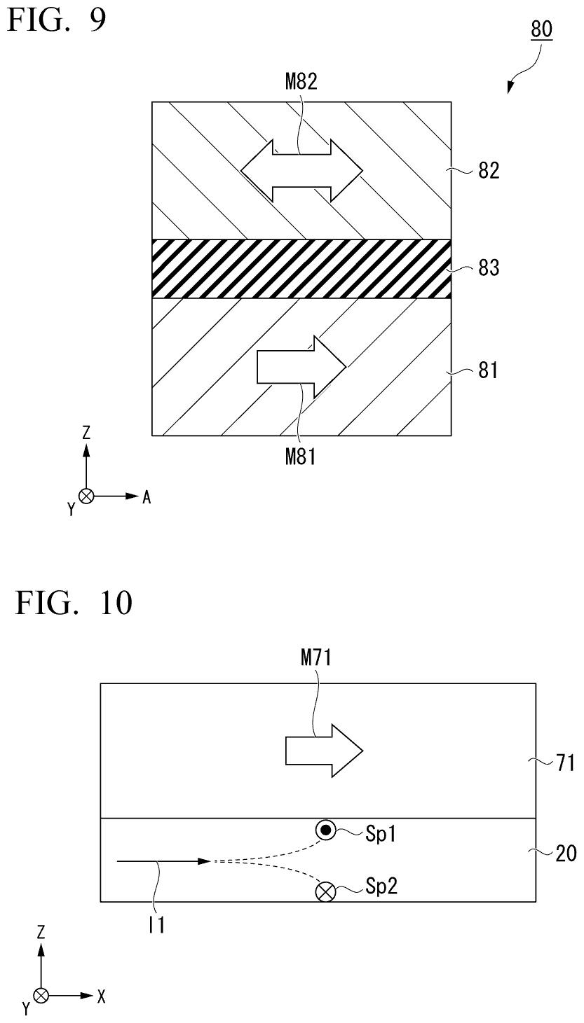

FIG. 9 is a sectional view of an example of the magnetic resistance changing element 80 in the hydrogen sensor 110 according to the sixth embodiment. The magnetic resistance changing element 80 is an example of a TMR element.

The magnetic resistance changing element 80 includes a reference layer 81, a free layer 82, and a non-magnetic layer 83. The non-magnetic layer 83 is sandwiched between the reference layer 81 and the free layer 82.

The reference layer 81 and the free layer 82 are ferromagnetic bodies. A material similar to that of the magnetic layer 71, for example, is used for the reference layer 81 and the free layer 82. The reference layer 81 includes a magnetization M81. The free layer 82 includes a magnetization M82. An orientation direction of the magnetization M81 of the reference layer 81 is less likely to change than the orientation direction of the magnetization M82 of the free layer 82 when a predetermined external force is applied. Once the orientation direction of the magnetization M71 of the magnetic layer 71 changes, the orientation direction of the magnetization M82 changes.

The orientation directions of the magnetization M81 of the reference layer 81 and the magnetization M82 of the free layer 82 preferably intersect the orientation direction of the magnetization M71 of the magnetic layer 71 in a state where an external magnetic field is not applied. If the relationship is satisfied, then the magnetic resistance changing element 80 can detect a slight change in orientation direction of the magnetization M71.

The non-magnetic layer 83 contains a non-magnetic body. In a case where the non-magnetic layer 83 is an insulator (in a case where the non-magnetic layer 83 is a tunnel barrier layer), it is possible to use, as a material thereof, Al₂O₃, SiO₂, MgO, and MgAl₂O₄, for example. In addition to these materials, materials obtained by replacing a part of Al, Si, or Mg with Zn, Be, or the like can also be used for the non-magnetic layer 83. Among these, MgO and MgAl₂O₄ are materials capable of realizing a coherent tunnel.

In a case where the magnetic resistance changing element 80 is a GMR element, the non-magnetic layer 83 is metal or a semiconductor. In a case where the non-magnetic layer 83 is metal, it is possible to use, as a material thereof, Cu, Au, Ag, or the like. Furthermore, in a case where the non-magnetic layer 83 is a semiconductor, it is possible to use, as a material thereof, Si, Ge, CuInSe₂, CuGaSe₂, Cu(In,Ga)Se₂, or the like.

Then, a hydrogen concentration detection method of the hydrogen sensor 110 will be described. The hydrogen concentration detection method of the hydrogen sensor 110 is different from those of the hydrogen sensors according to the first embodiment to the fifth embodiment.

Hydrogen contained in the measurement environment is adsorbed by the hydrogen adsorption layer 30 that is exposed to the measurement environment. Once hydrogen is adsorbed by the hydrogen adsorption layer 30, an electron state of the hydrogen adsorption layer 30 changes. The change in state of the hydrogen adsorption layer 30 leads to a change in characteristic of the magnetic layer 71. The characteristic of the magnetic layer 71 is, for example, magnetic anisotropy of the magnetization M71. For example, the magnetization M71 is likely to cause magnetization reversal in accordance with the amount of hydrogen adsorbed by the hydrogen adsorption layer 30.

The hydrogen concentration detection method of the hydrogen sensor 110 includes a current application process, a magnetization reversal detection process, and a hydrogen adsorption concentration detection process.

In the current application process, a pulse current is applied to the underlying layer 20 of the sensor element 70. The pulse current is applied until the magnetization M71 of the magnetic layer 71 is inverted with the current value increased.

Here, a principle that the magnetization M71 of the magnetic layer 71 is inverted by applying the pulse current to the underlying layer 20 of the sensor element 70 will be described. FIG. 10 is a diagram for explaining a function of the hydrogen sensor 110 according to the sixth embodiment.

Once a current I1 flows through the underlying layer 20, a spin flow is generated due to a spin Hall effect.

The spin Hall effect is a phenomenon that a spin current is induced in a direction (the Z direction, for example) perpendicularly intersecting a direction in which the current I1 flows on the basis of a spin orbital interaction in a case where the current I1 is caused to flow. The spin Hall effect is the same as the ordinary Hall effect in that a moving (transferring) direction of a moving (transferring) electrical charge (electrons) is bent. In the ordinary Hall effect, a moving direction of charged particles moving in a magnetic field is bent by a Lorentz force. On the contrary, the spin moving direction is bent merely by the electrons transferring (merely by a current flowing) even if the magnetic field is not present in the spin Hall effect.

Once the current I1 flows in the +X direction of the underlying layer 20 as illustrated in FIG. 10, for example, spin Sp1 polarized in the −Y direction is bent in the +Z direction with respect to an advancing direction, and spin Sp2 polarized in the +Y direction is bent in the −Z direction with respect to the advancing direction, for example.

The spin Sp1 is accumulated on the interface between the underlying layer 20 and the magnetic layer 71 and is injected to the magnetic layer 71.

The magnetization M71 of the magnetic layer 71 causes magnetization reversal by the spin Sp1 injected from the underlying layer 20. A current density of the current I1 necessary to cause magnetization reversal of the magnetization M71 differs depending on the amount of hydrogen adsorbed by the hydrogen adsorption layer 30. As the amount of hydrogen adsorbed by the hydrogen adsorption layer 30 increases, the magnetization M71 is inverted at a lower current density.

In the magnetization reversal detection process, reversal of the magnetization M71 is detected. Once the orientation of the magnetization of the magnetization M71 is inverted, a magnetic field in the measurement environment changes. The change in magnetic field is detected by the magnetic resistance changing element 80. With the change in magnetic field in the measurement environment, the magnetization orientation of the magnetization M82 of the free layer 82 in the magnetic resistance changing element 80 changes. Once a relative angle between the magnetization M82 of the free layer 82 and the magnetization of the reference layer 81 changes, the resistance value of the magnetic resistance changing element 80 in the Z direction changes. It is possible to detect reversal of the magnetization M71 of the magnetic layer 71 by measuring the resistance value of the magnetic resistance changing element 80 in the Z direction.

In the hydrogen adsorption concentration detection process, the adsorption concentration of hydrogen adsorbed by the hydrogen sensor 110 is obtained from the magnitude of the current value when the magnetization M71 of the magnetic layer 71 is inverted. A relationship between the hydrogen adsorption concentration and the magnetization reversal density is obtained in prior studies. It is possible to detect the hydrogen concentration in the measurement environment from the current density when the magnetization reversal occurs on the basis of the result obtained in the prior studies.

The hydrogen sensor 110 according to the sixth embodiment measures the amount of hydrogen in the measurement environment on the basis of a change in magnetization M71 of the magnetic layer 71 due to adsorption of hydrogen to the hydrogen adsorption layer 30. The hydrogen sensor 110 according to the present embodiment consumes a small amount of power. The hydrogen sensor 110 according to the present embodiment also operates at room temperature.

Also, the resistance of the hydrogen sensor 110 also changes in response to adsorption of methane or ethane. Performance of detecting methane or ethane is about 1/100 the performance of detecting hydrogen. Therefore, a influence of methane or ethane is sufficiently ignorable even in a case where hydrogen and methane or ethane are present at the same time in the measurement environment and in the environment at room temperature.

Also, although the example in which the magnetization M71 of the magnetic layer 71 is inverted by applying a current to the underlying layer 20 has been described here, a current may be applied to the hydrogen adsorption layer 30. Since the spin Hall effect also occurs in the hydrogen adsorption layer 30, the spin generated in the hydrogen adsorption layer 30 may be injected to the magnetic layer 71. In this case, the underlying layer 20 and the substrate 10 may not be provided.

The first embodiment to the sixth embodiment have been exemplified hitherto, and specific configurations of the hydrogen sensors have been described. The hydrogen sensor according to the present disclosure is not limited to these exemplified configurations, and various modifications can be made thereon within the scope in which the gist is satisfied. For example, the hydrogen sensor 110 according to the sixth embodiment may be provided with the heat absorber 51 or the heat generator 52.

### EXPLANATION OF REFERENCES

10, 11 Substrate
20, 21 Underlying layer
30, 31 Hydrogen adsorption layer
30A Particle
40 Intermediate layer
50, 60, 70 Sensor element
51 Heat absorber
52 Heat generator
71 Magnetic layer
80 Magnetic resistance changing element
81 Reference layer
82 Free layer
83 Non-magnetic layer
101, 102, 103, 104, 105, 110 Hydrogen sensor
M71, M81, M82 Magnetization
S1, S2, S4 First surface
S3 Surface
T1 First terminal
T2 Second terminal

What is claimed is:

1. A hydrogen sensor comprising:
a substrate;
an underlying layer with conductivity; and
a hydrogen adsorption layer with a resistance changing in response to adsorption of hydrogen,
wherein the underlying layer is located between the substrate and the hydrogen adsorption layer, and
a surface roughness of the hydrogen adsorption layer is smaller than a film thickness of the hydrogen adsorption layer.

2. The hydrogen sensor according to claim 1, further comprising:
a magnetic layer,
wherein the magnetic layer is located between the underlying layer and the hydrogen adsorption layer, and
magnetization of the magnetic layer is configured such that a magnetization direction changes in accordance with the amount of hydrogen adsorbed by the hydrogen adsorption layer.

3. The hydrogen sensor according to claim 1, wherein the hydrogen adsorption layer includes a plurality of particles dispersed in a plane when seen in a stacking direction.

4. The hydrogen sensor according to claim 1, wherein a surface of the underlying layer on a side far from the substrate includes unevenness projecting or recessed in a stacking direction.

5. The hydrogen sensor according to claim 4, wherein a surface of the substrate on the side of the underlying layer includes unevenness projecting or recessed in the stacking direction.

6. The hydrogen sensor according to claim 4, further comprising:
an intermediate layer between the substrate and the underlying layer,
wherein a surface of the intermediate layer on the side of the underlying layer includes unevenness projecting or recessed in the stacking direction.

7. The hydrogen sensor according to claim 1, wherein a resistance changing portion including the underlying layer and the hydrogen adsorption layer has a meander wiring structure.

8. The hydrogen sensor according to claim 1, further comprising:
a heat absorber,
wherein the heat absorber is not in direct electrical contact with the underlying layer and the hydrogen adsorption layer.

9. The hydrogen sensor according to claim 1, further comprising:
a heat generator,
wherein the heat sensor is not in direct electrical contact with the underlying layer and the hydrogen adsorption layer.

10. The hydrogen sensor according to claim 1, wherein a thickness of the hydrogen adsorption layer is less than 2 nm.

11. The hydrogen sensor according to claim 1, wherein a thickness of the underlying layer is equal to or less than 3 nm.

12. The hydrogen sensor according to claim 1, wherein a hydrogen adsorption material constituting the hydrogen adsorption layer is metal, an alloy, an oxide, a nitride, or an oxynitride containing any one selected from a group consisting of Mg, Al, Ti, Fe, Zr, Pd, Pt, Hf, and Ta.

13. The hydrogen sensor according to claim 1, wherein the underlying layer has a higher resistivity than the hydrogen adsorption layer.

14. The hydrogen sensor according to claim 1, wherein the underlying layer contains any one selected from a group consisting of NiCr, β-W, Zr, Cr, Ta, alumel, chromel, Ti, NbN, TaN, TiN, VN, and ZrN.

15. The hydrogen sensor according to claim 2, further comprising:
a magnetic resistance changing element,
wherein the magnetic resistance changing element is not electrically connected to the magnetic layer, and
the magnetic resistance changing element is configured to be able to detect a change in magnetization direction of the magnetic layer.

16. The hydrogen sensor according to claim 15, wherein the magnetic resistance changing element is any one selected from a group consisting of an AMR element, a GMR element, a TMR element, and an AHE element.

17. The hydrogen sensor according to claim 15, wherein an orientation of magnetization of the magnetic layer and an orientation of magnetization of a ferromagnetic layer of the magnetic resistance changing element intersect with each other.

18. A hydrogen detection method comprising:
applying a pulse current to the underlying layer in the hydrogen sensor according to claim 2 while raising a current value;
detecting, with a magnetic resistance changing element, that magnetization of the magnetic layer in the hydrogen sensor has been inverted; and
obtaining an adsorption concentration of hydrogen adsorbed by the hydrogen sensor from a magnitude of a current value when the magnetization of the magnetic layer has been inverted.
